(19) United States
(12) Patent Application Publication
(10) Pub. No.: US 2022/0260543 A1
(43) Pub. Date: Aug. 18, 2022

(54) REAL-TIME ATMOSPHERIC DIFFUSION MONITORING SYSTEM

(71) Applicant: TAESUNG ENVIRONMENTAL RESEARCH INSTITUTE CO., LTD., Ulsan (KR)

(72) Inventors: Gi Yeol YUN, Ulsan (KR); Seok Man KIM, Ulsan (KR)

(21) Appl. No.: 17/618,211

(22) PCT Filed: Dec. 9, 2019
(86) PCT No.: PCT/KR2019/017299

§ 371 Date: Dec. 10, 2021

(30) Foreign Application Priority Data
Oct. 31, 2019 (KR) 10-2019-0137077

(51) Int. Cl. G01N 33/00; G01W 1/02

(52) U.S. Cl. CPC G01N 33/0075; G01W 1/02; G06N 20/20; G01N 2033/0068; G01N 33/0062

(57) ABSTRACT

According to one embodiment of the present disclosure, provided is a real-time atmospheric diffusion monitoring system comprising: a fixed odor measuring device which is fixed at a specific point and measures smell information; a vehicle odor measuring device which measures smell information while moving on the ground; a drone which measures smell information while moving in the air; and a server which analyzes and manages odor information spreading in the atmosphere on the basis of the smell information collected from at least one of the fixed odor sensing device, the vehicle odor sensing device, and the drone.

### TECHNICAL FIELD

The present invention relates to a real-time atmospheric diffusion monitoring system. More specifically, the present invention relates to a system for analyzing and managing odor and weather information diffused in the air on the basis of odor information collected from at least one of a fixed odor measuring device, a vehicular odor measuring device and a drone.

### BACKGROUND ART

As the industry develops, the influence of odor generated from industrial complexes on the surrounding areas is becoming a social issue. Accordingly, the government enacted the odor prevention act and has legally controlled the amount of generated odor since 2005.

The diffusion degree of odor generated from pollution sources is determined by a terrain or an atmospheric condition, etc. When odor is generated from a specific point, in order to accurately track odor generating sources which affect the odor generation, it is necessary to obtain accurate information on atmospheric conditions, etc., at the time of odor generation. The atmospheric conditions can be measured if enough atmospheric measuring networks are set up. In addition, in order to backtrack the odor generating sources, it is necessary to obtain information on main pollutants produced from the odor generating sources, most of which has been secured by inspecting the process of the odor generating sources, etc.

In this situation, the most important information for the backtracking of the odor generating sources is component analysis of pollutants included when the odor is generated. For accurate component analysis, it is necessary to collect the gas at the time of odor generation in real time.

However, now, odor handling employees irregularly visit the area where civil complaints about odor generation often arise, carrying a simple portable device for collecting the air to collect the air manually. Odor tends to instantaneously appear and disappear due to atmospheric conditions, etc., which makes it difficult to collect the gas for accurate analysis.

Furthermore, the degree of sensing odor varies depending on individual's sense of smell, and the diffusion degree of odor is affected by atmospheric conditions, etc. Thus, for effective analysis, it is essential to accurately measure the concentration of odor and collect in real time the gas at the time of odor generation at the site upon odor generation.

However, the gas collecting at the site at the moment of initial stage of odor management depends on humans. That is, since odor managers visit the site and collect the gas on their own, they fail to collect the gas at the exact time of odor generation due to space/time constraints, resulting in inaccurate odor analysis, etc. As such, there are many problems in odor management.

### SUMMARY OF INVENTION

### Technical Task

The present invention is to solve the above-described problems of the prior art. It is an object of the present invention to provide a system for analyzing and managing information on odor diffused in the air on the basis of smell information collected from at least one of a fixed odor sensing device, a vehicular odor sensing device and a drone.

The object of the present invention is not limited to the aforementioned objects, and other objects that are not mentioned can be clearly understood from the following description.

### Means for Solving the Task

According to an embodiment of the present invention, provided is a real-time atmospheric diffusion monitoring system, comprising a fixed odor measuring device for measuring odor information while being fixed at a specific point; a vehicular odor measuring device for measuring odor information while moving on the ground; a drone for measuring odor information while moving in the air; and a server for analyzing and managing information on odor diffused in the air on the basis of the odor information collected from at least one of the fixed odor sensing device, the vehicular odor sensing device and the drone.

The server may confirm a tendency of odor diffusion using actual weather conditions and three-dimensional wind fields through an odor diffusion modeling program.

The server may analyze the information on odor diffused in the air using a CALPUFF modeling technique showing a complex terrain and a change in wind field.

The server may classify smell and odor and analyze characteristics of each of smell and odor through a smell prediction program.

The server may classify types and intensities of the odor information through a multinomial logistic regression (MLR) model.

The server may predict dilution factors of the odor information through a gaussian linear regression (GLR) model.

The real-time atmospheric diffusion monitoring system according to an embodiment of the present invention may comprise a fixed odor measuring device for measuring smell information while being fixed at a specific point; a vehicular odor measuring device for measuring smell information while moving on the ground; a drone for measuring smell information while moving in the air; and a server for analyzing and managing information on odor diffused in the air on the basis of the odor information collected from at least one of the fixed odor sensing device, the vehicular odor sensing device and the drone.

Also, the server may confirm a tendency of odor diffusion using actual weather conditions and three-dimensional wind fields through an odor diffusion modeling program.

Also, the server may analyze the information on odor diffused in the air using a CALPUFF modeling technique showing a complex terrain and a change in wind field.

Also, the server may classify smell and odor and analyze characteristics of each of smell and odor through a smell prediction program.

Also, the server may classify types and intensities of the odor information through a random forest model.

Also, the server may analyze the information on odor diffused in the air by granting different reliabilities to a first odor information obtained from the fixed odor measuring device, a second odor information obtained from the vehicular odor measuring device and a third odor information obtained from the drone.

Also, the server may grant a highest reliability to the first odor information, a medium reliability to the second odor information and a lowest reliability to the third odor information.

Also, the server may increase the reliability of the third odor information when an air current measured at a position of the drone is a descending air current, and decrease the reliability of the third odor information when an air current measured at a position of the drone is an ascending air current.

### Effect of Invention

According to an embodiment of the present invention, a way of reducing odor can be easily established by measuring or collecting an odor substance generated from a specific point in real time with an odor measuring device and an odor collecting equipment for analysis, and identifying an odor causing substance.

The effects of the present invention are not limited to the above-mentioned effects, and it should be understood that the effects of the present invention include all effects that could be inferred from the configuration of the invention described in the detailed description of the invention or the appended claims.

### BRIEF DESCRIPTION OF DRAWINGS

FIG. 1 is a view illustrating an integrated monitoring system for odor tracking according to an embodiment of the present invention;

FIG. 2 is a view illustrating a system block diagram of the integrated monitoring system for odor tracking according to an embodiment of the present invention;

FIG. 3 is a view illustrating a network block diagram of the integrated monitoring system for odor tracking according to an embodiment of the present invention;

FIG. 4 is a view illustrating a flow of collecting odor data according to an embodiment of the present invention;

FIG. 5 is a view illustrating the screen on which an odor diffusion modeling program according to an embodiment of the present invention is run;

FIG. 6 is a view illustrating the running mechanism of a smell prediction program according to an embodiment of the present invention;

FIG. 7 is a view illustrating an example of obtaining odor related data using big data and an odor monitoring system (OMS) according to an embodiment of the present invention; and

FIG. 8 is a view illustrating an example of an OMS according to an embodiment analyzing odor.

### DETAILED MEANS FOR CARRYING OUT THE INVENTION

Hereinafter, the present invention will be explained with reference to the accompanying drawings. The present invention, however, may be modified in various different ways, and should not be construed as limited to the embodiments set forth herein. Also, in order to clearly explain the present invention in the drawings, portions that are not related to the present invention are omitted, and like reference numerals are used to refer to like elements throughout the specification.

Hereinafter, embodiments of the present invention will be explained in more detail with reference to the accompanying drawings.

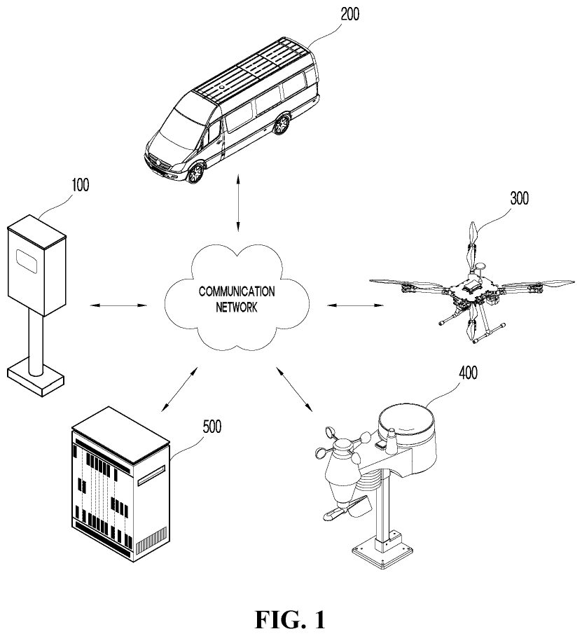

FIG. 1 is a view illustrating an integrated monitoring system for odor tracking according to an embodiment of the present invention.

Referring to FIG. 1, the integrated monitoring system for odor tracking may comprise a fixed odor measuring device 100, a vehicular odor measuring device 200, an odor sensing and collecting drone 300, a whether measuring device 400, and a server 500, which can communicate with each other through a communication network.

First, the communication network may include various communication networks, such as a local area network (LAN), a metropolitan area network (MAN), a wide area network (WAN), a mobile communication network, etc., regardless of communication aspects such as wired and wireless communications, etc.

The fixed odor measuring device 100 may measure and analyze odor information while being fixed at a specific point and collect odor information.

The vehicular odor measuring device 200 may measure and analyze an odor causing substance while moving on the ground and collect odor information.

The odor sensing and collecting drone 300 may measure and analyze an odor causing substance while moving in the air and collect odor information.

Each of the fixed odor measuring device 100, vehicular odor measuring device 200 and odor sensing and collecting drone 300 may sense an odor causing substance in real time and transmit odor information to the server 500 when sensing an odor causing substance.

The weather measuring device 400 may measure and collect weather information.

The server 500 may receive odor information collected from the fixed odor measuring device 100, vehicular odor measuring device 200 and odor sensing and collecting drone 300, etc., and analyze and manage information on odor generated from a specific point on the basis of the odor information collected from various devices.

The server 500 may convert and compute at least one of a smell type, a smell intensity, a complex odor and an odor causing substance concentration using the odor information.

The server 500 may receive weather information collected from the weather measuring device 400, and compare the weather information and odor information to analyze a pattern of odor generation.

The server 500 may predict odor generation from the pattern of odor generation and provide predicted odor information according to the result of prediction of odor generation.

* The server 500 may send an alert notification message of odor generation to a manager terminal (not illustrated) when deciding that odor is generated as a result of analysis of odor information.

According to an embodiment, the integrated monitoring system for odor tracking measures a smell type, a smell intensity, a complex odor and an odor causing substance concentration in real time, and may accordingly enable quick preparation of a measure in response to civil complaints when civil complaints about odor arise.

The integrated monitoring system for odor tracking may be classified into the fixed odor measuring device 100 for sensing odor causing substances in real time and transmitting information to the server 500 and the server 500 for receiving the information and displaying the same.

The integrated monitoring system for odor tracking may in real time store in database measurement data of an odor sensor which is measured at the site.

The integrated monitoring system for odor tracking may be classified into odor measuring devices such as the fixed odor measuring device 100, vehicular odor measuring device 200 and odor sensing and collecting drone 300, a weather measuring device such as the weather measuring device 400, and the server 500. Data transmission between the odor measuring devices and the server 500 may be carried out wirelessly. An odor measurement result measured at a point where the odor measuring device is positioned may be transmitted to the server 500 to be displayed.

The fixed odor measuring device 100, vehicular odor measuring device 200 or odor sensing and collecting drone 300 may transmit the measured odor measurement result to the server 500. At this time, a transmission frequency of transmitting the odor measurement result to the server 500 may be determined differently depending on situations. The transmission frequency may vary according to the odor measurement result and odor measurement location. For example, the transmission frequency may be determined based on how high a smell intensity, concentration or dilution factor is according to the odor measurement result. As the smell intensity, concentration or dilution factor increases, the transmission frequency may stepwise increase. As another example, the transmission frequency may be high when a change of a predetermined value or more is expected at the current odor measurement location in a predetermined time. For example, the transmission frequency may be high when a dramatic change in the odor measurement result is expected at the current odor measurement location based on weather conditions such as wind, etc., and surrounding odor generation conditions. The transmission frequency may be determined according to a size of the expected change.

The server 500 may determine the location of odor generation by using odor information received from the fixed odor measuring device 100, vehicular odor measuring device 200 and odor sensing and collecting drone 300, and weather information received from the weather measuring device 400. The server 500 may process the odor information received from the fixed odor measuring device 100, vehicular odor measuring device 200 and odor sensing and collecting drone 300 in different ways and use the information, in order to determine the location of odor generation.

For example, the server 500 may grant different reliabilities to odor information received from the fixed odor measuring device 100, vehicular odor measuring device 200 and odor sensing and collecting drone 300. The reliabilities of hardware for odor measurement mounted on the fixed odor measuring device 100 and mobile odor measuring device 200 may be higher than the reliability of hardware for odor measurement mounted on the odor sensing and collecting drone 300. As such, the server 500 may perform integrated monitoring for odor tracking by granting a high weight value to the odor information received from the fixed odor measuring device 100 and vehicular odor measuring device 200 and granting a low weight value to the odor sensing and collecting drone 300.

As another example, the server 500 may perform odor monitoring by reflecting characteristics of hardware for odor measurement included in the fixed odor measuring device 100, vehicular odor measuring device 200 and odor sensing and collecting drone 300. As an example, when hardware for odor measurement with high reliability in monitoring hydrogen sulfide is mounted on the fixed odor measuring device 100, hardware for odor measurement with high reliability in monitoring ammonia is mounted on the vehicular odor measuring device 200, and hardware for odor measurement with high reliability in monitoring complex odor is mounted on the odor sensing and collecting drone 300, the server 500 may perform integrated monitoring for odor tracking (for example, determining the location of odor generation) by granting a highest weight value to the odor information obtained from the fixed odor measuring device 100 when performing the monitoring of hydrogen sulfide, granting a highest weight value to the odor information obtained from the vehicular odor measuring device 200 when performing the monitoring of ammonia, and granting a highest weight value to the odor information obtained from the odor sensing and collecting drone 300 when performing the monitoring of complex odor.

As another example, the server 500 may apply time difference to odor information received from the odor sensing and collecting drone 300 when performing integrated monitoring for odor tracking. When an altitude which is a reference altitude when performing monitoring of odor is close to the ground, time difference may exist in order for odor information measured at a position of high altitude to be reflected in a position of low altitude. Accordingly, the server 500 may use weather information received from the weather measuring device 400 to determine whether the air current at the position of the odor sensing and collecting drone 300 is an ascending air current or a descending air current, and determine an intensity of the air current. The server 500 according to an embodiment may reflect odor information received from the odor sensing and collecting drone 300 at a rate lower than the predetermined rate (for example, 5%), when the air current at the position of the odor sensing and collecting drone 300 is an ascending air current. Or, the server 500 according to an embodiment may perform monitoring of odor on the ground by reflecting odor information received from the odor sensing and collecting drone 300 at a time interval which is inversely proportional to the intensity of the air current, when the air current at the position of the drone 300 is a descending air current.

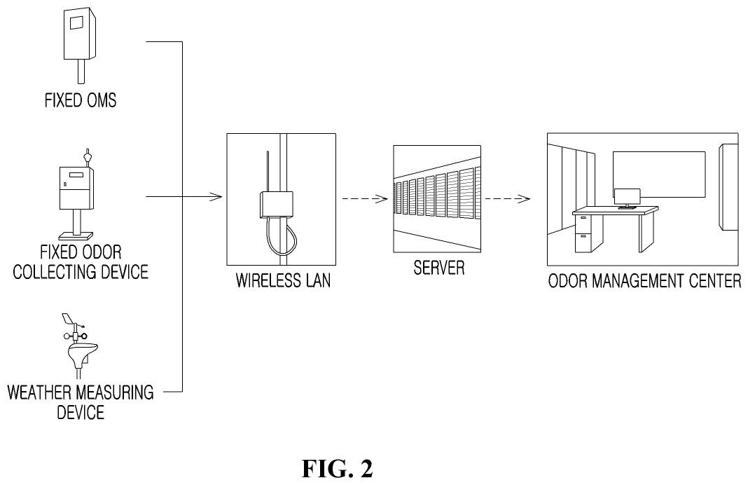

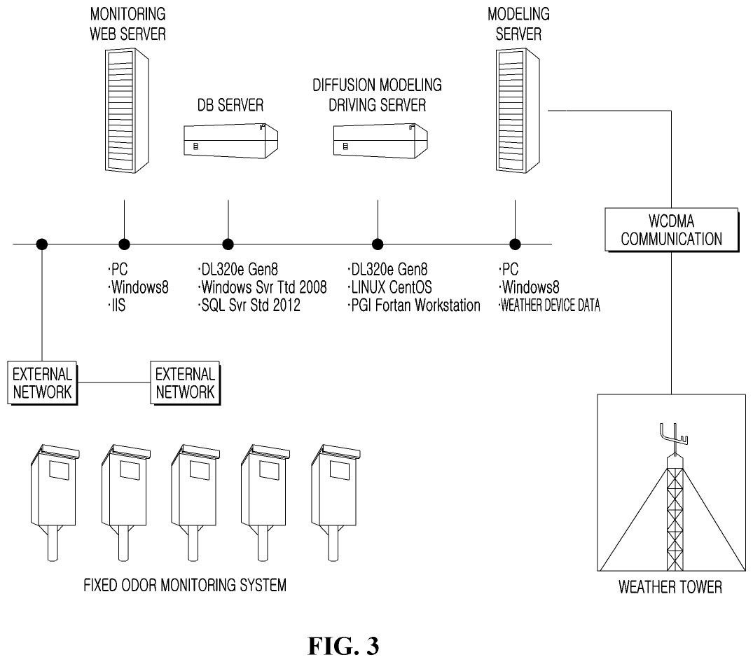

FIG. 2 is a view illustrating a system block diagram of the integrated monitoring system for odor tracking according to an embodiment of the present invention, and FIG. 3 is a view illustrating a network block diagram of the integrated monitoring system for odor tracking according to an embodiment of the present invention.

As illustrated in FIG. 2 and FIG. 3, the integrated monitoring system for odor tracking may analyze and manage data on surrounding odor by measuring in real time main odor causing substances (for example, complex odor, hydrogen sulfide, ammonia, TVOCs, etc.) generated from a specific point or national industrial complexes in which odor emitting companies are concentrated and weather information (wind direction, wind speed, temperature, humidity, etc.), and transmitting collected data (smell intensity, concentration, diffusion path, weather information, etc.) to a control system implemented into the server 500 remotely, using a wireless communication network (WCDMA, LTE, etc.).

The integrated monitoring system for odor tracking may configure an unmanned odor collecting device as an integral type and a separate type according to consumer's demands, automatically collect a sample in steps when exceeding an odor reference value, and provide a function allowing a manager to remotely collect odor from the site at any time.

The integrated monitoring system for odor tracking may automatically send a text message of alert and state to a manager using SMS and APP when odor is generated and odor of a threshold value or more is generated.

As for the integrated monitoring system for odor tracking, an unmanned odor collecting system and a weather measuring system may be manufactured as an integral type and a separate type according to options.

The integrated monitoring system for odor tracking includes an odor sensing device and an information processing system. The weather measuring device 400 may analyze the generation pattern by collecting weather information and comparing the information with odor information, and may be implemented into an integrated odor information management system enabling preparation of a measure of predicting and preventing odor generation by displaying the sensed and measured odor information outside in real time or periodically.

The integrated monitoring system for odor tracking may provide total condition services regarding odor, monitor odor in real time using smartphone applications and PC, confirm the surrounding fine dust level by interconnecting with weather information and national networks, and enable an immediate response upon event occurrence through prediction and alert notification.

As a method for collecting odor data, odor collected from an odor causing source and weather data are transmitted to a signal converter, and the odor and weather signal converter may convert the collected analog signal to a digital signal, and process a physical signal with the smell type, smell intensity and concentration to transmit the signal to a data analyzer.

The odor data analyzer may process the data collected from the signal convert in various forms and store the same in a storing device in the analyzer.

The analysis data of the odor measuring device may include real-time data, odor intensity data, odor diffusion three-dimensional data, etc.

The real-time data is real-time odor data measured by an automatic odor measuring device, and with the measurement data, for example, a concentration of odor per second may be analyzed in real time.

The odor intensity data is data measured by an automatic odor measuring device on smell intensity, smell type, concentration and dilution factor for each gas with respect to measurement ranges and odor intensities, and as for the odor intensity data, measurement data may be stored in order to send an alert text message and display odor modeling when odor of a threshold value or more is generated.

The odor diffusion three-dimensional data is three-dimensional data made by the server 500 through a modeling program by processing actual odor into a signal when odor of a predetermined value or more is generated, and then storing the signal as a file, and when a file of the measured odor information is created, abnormal odor data is stored in a management program, and the created file may be stored along with data on the smell intensity, smell type, concentration, dilution factor, etc.

As a method for analyzing odor data, the odor data may be processed and analyzed by odor data processing S/W of the odor analyzer on the basis of odor data collected from the odor measuring device by the signal converter.

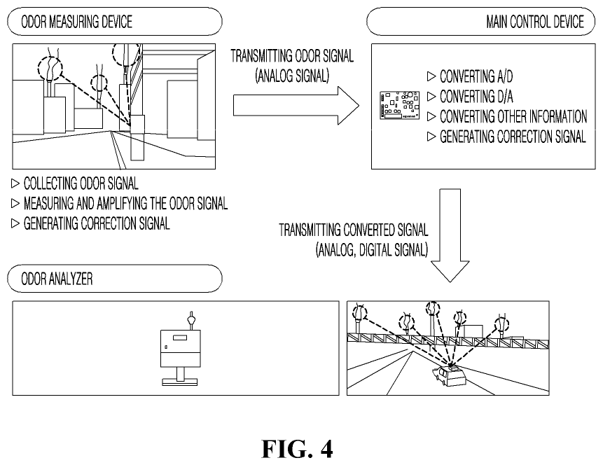

FIG. 4 is a view illustrating a flow of collecting odor data according to an embodiment of the present invention.

As illustrated in FIG. 4, the odor measuring device may collect an odor signal, perform measurement and amplification of the odor signal, generate a correction signal, and convert the odor signal into a digital signal for transmission.

The main control device may perform a process of sampling, A/D conversion, D/A conversion, other information conversion, correction signal generation, etc. on the odor signal, and transmit a signal converted into an analog signal or a digital signal to the odor analyzer.

As for the measurement data transmitted in real time from the automatic odor measuring device and the measurement data returned by a request of a communication server, an end of transmission (EOT) signal is transmitted to notify a management system communication server of completion of transmission when transmission is terminated.

The transmission and reception data is filled from the right side of the number of digits of a format defined by the communication protocol, and when no data is present or the data is a fixed number of digits or less, a blank value may be filled therein.

The transmission side transmits the last data and receives an EOT signal from the reception side, and then transmission is terminated. Upon completion of transmission, the connection may be closed.

As a way of transmitting odor data, TCP/IP is used for transmission and reception with a management center. When the automatic odor measuring device transmits data to the management center, the management center may be the server 500. When the management center transmits a telecommand to the odor measuring device, the odor measuring device may be the server 500.

According to an embodiment of the present invention, the server 500 may receive smell information collected from the fixed odor measuring device 100, vehicular odor measuring device 200, odor sensing and collecting drone 300, etc., and analyze and manage information of odor diffused in the air on the basis of the smell information collected from various devices.

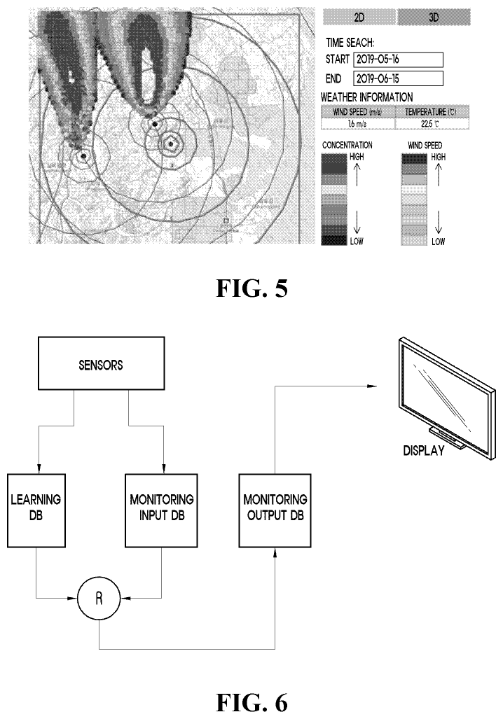

FIG. 5 is a view illustrating the screen on which an odor diffusion modeling program according to an embodiment of the present invention is run.

As illustrated in FIG. 5, the server 500 may confirm a tendency of odor diffusion using actual weather conditions and three-dimensional wind fields through an odor diffusion modeling program, and analyze the information on odor diffused in the air using a CALPUFF modeling technique showing a complex terrain and a change in wind field.

The server 500 may analyze information on odor diffused in the air on the basis of the direction of wind, current temperature, distribution of odor, surrounding terrain and surrounding facilities. For example, the server 500 may analyze information on diffused odor by comprehensively considering surrounding facilities (for example, whether specific factories are operating) and the terrain of the surrounding facilities (for example, the mountains). As another example, the server 500 may raise the height of altitude considering diffusion to the predetermined value or more if an ascending air current occurs at the moment, and lower the height of altitude considering diffusion to the predetermined value or less if a descending air current occurs at the moment.

The server 500 may reduce the minimum unit of wind that is used for confirming a tendency of odor diffusion when the odor intensity is a threshold value or more or when the odor type is a predetermined type.

The confirmation frequency of confirming the tendency of odor diffusion may be determined differently depending on situations. The confirmation frequency may vary according to the odor measurement result and odor measurement location. For example, the confirmation frequency may be determined based on how high the smell intensity, concentration or dilution factor is according to the odor measurement result. As the smell intensity, concentration or dilution factor increases, the confirmation frequency may stepwise increase.

Also, the confirmation frequency may be determined differently depending on positions on the overall map. For an area where a change of a predetermined value or more is expected within a predetermined time (for example, in real time), the confirmation frequency may be increased. For example, for an area where a dramatic change in the odor measurement result is expected at the current odor measurement location based on weather conditions such as wind, etc., and surrounding odor generation conditions, the confirmation frequency may be higher than other areas. The confirmation frequency may be determined according to the size of the expected change.

For example, when the wind is strong, it is seen that a dramatic change in the odor measurement result is expected, and the confirmation frequency may be determined to be relatively high. The server 500 may determine the confirmation frequency such that the average wind strength of the corresponding area and the confirmation frequency of the corresponding area are proportional to each other.

As another example, when there is a great difference between a maximum value and a minimum value of the odor concentration in an area within a specific range, it is seen that a dramatic change in the odor measurement result is expected, and the confirmation frequency may be determined to be relatively high. The server 500 may determine the confirmation frequency such that the difference between the maximum value and the minimum value of the odor concentration in the corresponding area and the confirmation frequency of the corresponding area are proportional to each other. The size of the corresponding area may be a predetermined value. For example, the server 500 may determine the confirmation frequency to correspond to the difference between the maximum value and the minimum value of the odor concentration within 1 [ha], with 1 [ha] as a unit area.

As another example, when there is a great difference between a maximum value and a minimum value of the temperature in an area within a specific range, it is seen that a dramatic change in the odor measurement result is expected, and the confirmation frequency may be determined to be relatively high. The server 500 may determine the confirmation frequency such that the difference between the maximum value and the minimum value of the temperature in the corresponding area and the confirmation frequency of the corresponding area are proportional to each other.

The server 500 may determine a moving path of the vehicular odor measuring device 200 according to road conditions and odor information, etc. Since the vehicular odor measuring device 200 basically moves on the road, the moving path of the vehicular odor measuring device 200 may be determined on the basis of the conditions of the road (for example, the location of the road, traffic conditions, etc.). For example, in the case of a road with heavy traffic, relatively slow driving is expected, and thus the road may have a relatively low priority to be selected as a moving path. As another example, the server 500 may determine the moving path of the vehicular odor measuring device 200 to go via the roads around an expected odor generation area (for example, location of factory chimney).

The server 500 may determine an expected odor generation area, and determine a road around the expected odor generation area as a moving path of the vehicular odor measuring device 200 when there is a road around the expected odor generation area. When there is no road around the expected odor generation area, the server 500 may determine a priphery of the expected odor generation area as a moving path of the drone 300. In order to obtain odor information from the expected odor generation area, the server 500 may prioritize an approach of the vehicular odor measuring device 200 to an approach of the drone 300. Since the reliability of hardware mounted on the vehicular odor measuring device 200 is higher than the reliability of the drone 300 and the vehicular odor measuring device 200 obtains odor information on the ground unlike the drone 300, for an area where odor is expected to be generated (for example, an area having a probability of odor generation of a predetermined value or more), the server 500 may determine the moving paths of the vehicular odor measuring device 200 and the drone 300 such that the approach of the vehicular odor measuring device 200 has priority over the approach of the drone 300.

Also, the server 500 may determine the moving path of the vehicular odor measuring device 200 in consideration of the direction of wind around the expected odor generation area. For example, when an east wind blows in the expected odor generation area, the server 500 may determine the moving path of the vehicular odor measuring device 200 such that an east point of the expected odor generation area and a west point of the expected odor generation area fall within the moving path of the vehicular odor measuring device 200. The server 500 obtains odor information from both of the point in the direction from which wind blows and the point in the direction to which wind blows with respect to the expected odor generation area, and can clearly confirm whether odor is really generated from the expected odor generation area.

The server 500 may determine the moving path of the odor sensing and collecting drone 300 in consideration of the direction of wind around the expected odor generation area. For example, when an east wind blows in the expected odor generation area, the server 500 may determine the moving path of the odor sensing and collecting drone 300 such that the odor sensing and collecting drone 300 moves from an east point of the expected odor generation area to a west point of the expected odor generation area. The server 500 obtains odor information continuously on the line which connects the point in the direction from which wind blows and the point in the direction to which wind blows with respect to the expected odor generation area, and can clearly confirm whether odor is really generated from the expected odor generation area.

FIG. 6 is a view illustrating the running mechanism of a smell prediction program according to an embodiment of the present invention.

As illustrated in FIG. 6, the server 500 may classify smell and odor and analyze characteristics of smell and odor through a smell prediction program.

That is, the server 500 may classify the measured smell and odor on the basis of data in the database of an object for analysis through a smell prediction program, and predict types, intensities and dilution factors of smell and odor using algorisms that can analyze characteristics of each of them.

The server 500 may classify the types and intensities of smell information employing random forest based machine learning and artificial intelligence, and predict dilution factors of smell information by fusing real-time data and accumulated data (big data).

Regarding random forest based machine learning and artificial intelligence for classifying the types and intensities of smell information, temperature, humidity and sensor data input to learning database may be used as independent variables for model generation. Patterns may be classified into classes on the basis of the types and intensities. The classified class values may be stored and displayed as predictive values. A class value having the highest probability may be stored and displayed as a predictive value by estimating the probability of belonging to each class with dependent variables.

In particular, the smell intensity and dilution factor are consistent with Weber-Fechner's law, and the law may be applied to the model generating and predicting process. The smell intensity may be calculated by a formula such as “a+K*log(dilution factor).”

As such, according to an embodiment of the present invention, a way of reducing odor can be easily established by measuring or collecting an odor substance generated from a specific point in real time with an odor measuring device and an odor collecting equipment for analysis, and identifying an odor causing substance.

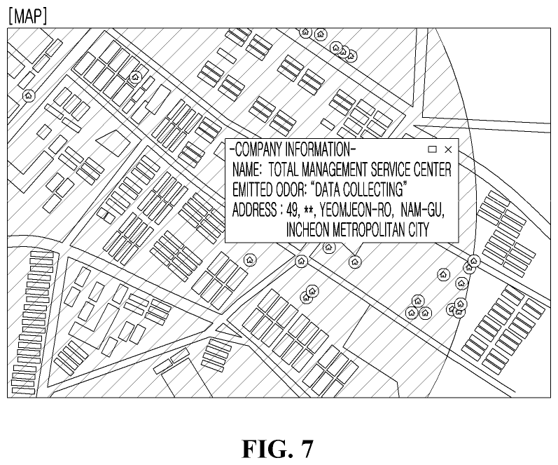

FIG. 7 is a view illustrating an example of obtaining odor related data using big data and an odor monitoring system (OMS) according to an embodiment of the present invention.

The server 500 according to an embodiment may establish big data. For example, the server 500 may establish big data including all of information on factories involved in odor, weather information, information on odor in the air, measurement information on odor, etc. The information on factories involved in odor may include location information about factories, odor information that factories are expected to emit, time when factories emit odor substances, types of odor substances that were emitted by factories in the past, etc. The server 500 may establish big data including various information related to odor to determine a point which is the source of odor in real time. For example, the server 500 may use big data to determine an odor source point that is expected to affect the location where civil complaints about odor are filed when the civil complaints about odor are filed.

The server 500 and/or OMS may classify the types and intensities of smell information employing random forest based machine learning and artificial intelligence, and predict dilution factors of smell information by fusing real-time data and accumulated data (big data).

Regarding random forest based machine learning and artificial intelligence for classifying the types and intensities of smell information, temperature, humidity and sensor data input to learning database may be used as independent variables for model generation. Patterns may be classified into classes on the basis of the types and intensities. The classified class values may be stored and displayed as predictive values. A class value having the highest probability may be stored and displayed as a predictive value by estimating the probability of belonging to each class with dependent variables.

In particular, the smell intensity and dilution factor are consistent with Weber-Fechner's law, and the law may be applied to the model generating and predicting process. The smell intensity may be calculated by a formula such as “a+K*log(dilution factor).”

As such, according to an embodiment of the present invention, a way of reducing odor can be easily established by measuring or collecting an odor substance generated from a specific point with an odor measuring device and an odor collecting equipment in real time for analysis, and identifying an odor causing substance.

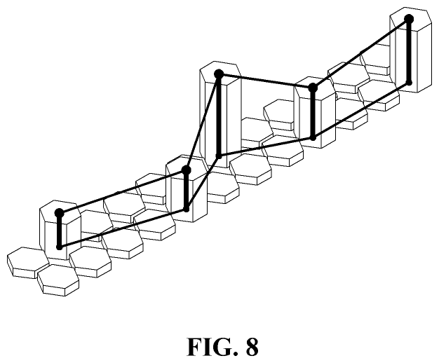

FIG. 8 is a view illustrating an example of an OMS according to an embodiment analyzing odor.

The OMS according to an embodiment may obtain and analyze odor information. For example, the OMS may analyze odor and specifically determine components included in the odor and concentrations of the components, etc. The OMS may comprise a plurality of sensors and analyze odor according to the degree of response of each sensor. For example, the OMS may obtain two-dimensional pattern types shown by the plurality of sensors according to the degree of response of the plurality of sensors arranged in two dimension, and determine causing substances and concentrations of the causing substances according to the obtained two-dimensional pattern types. For example, in the case of garlic smell, methyl acrylate may be 30 ppm, and ethyl acrylate may be 2 ppm. As another example, in the case of suffocating pungent smell, propenylbenzene may be 25 ppm, and NH3 may be 8 ppm.

As such, the OMS may comprise a plurality of sensors arranged in two dimension which show different patterns for each smell, and learn a relationship between the types of odor and the patterns of the plurality of sensors arranged in two dimension. For example, the obtained odor is analyzed using Sift-MS to obtain a result thereof, the OMS learns the analyzed result, and thereby the OMS may analyze odor. In this case, although the OMS is much lighter hardware than the Sift-MS, it may perform accurate odor analysis using the learning result through the Sift-MS.

The above-described description of the present invention is intended for illustration, and a person having ordinary knowledge in the art to which the present invention pertains will understand that the present invention may be easily modified in other specific forms without changing the technical spirit or essential features of the present invention. Therefore, it should be understood that the embodiments described above are exemplary in all respects and not restrictive. For example, each component described as a single type may be implemented in a distributed manner, and similarly, components described as distributed may be implemented in a combined form.

The scope of the present invention is defined by the accompanying claims. It should be construed that all modifications and embodiments derived from the meaning and scope of the claims and their equivalents fall within the scope of the present invention.

Meanwhile, the above-described method can be written as a program that can be executed in a computer, it can be implemented in a general-purpose digital computer to operate the program using a computer-readable recording medium. In addition, the structure of the data used in the above-described method can be recorded on the computer-readable recording medium through various means. The computer-readable recording medium may include a storage medium such as a magnetic storage medium (for example, a ROM, a RAM, a USB, a floppy disk, a hard disk, etc.) and an optical reading medium (for example, a CD-ROM, a DVD, etc.).

A person having ordinary knowledge in the art to which the present embodiment pertains will appreciate that the present invention may be embodied in a modified form without departing from the essential characteristics of the above description. Therefore, the disclosed methods should be considered in descriptive sense and not for purposes of limitation. The scope of the present invention is shown in the claims rather than the foregoing description, and all differences within the scope will be construed as falling within the present invention.

What is claimed is:

1. A real-time atmospheric diffusion monitoring system, comprising:
a fixed odor measuring device for measuring smell information while being fixed at a specific point;
a vehicular odor measuring device for measuring smell information while moving on the ground;
a drone for measuring smell information while moving in the air; and
a server for analyzing and managing information on odor diffused in the air on the basis of the odor information collected from at least one of the fixed odor sensing device, the vehicular odor sensing device and the drone.

2. The system of claim 1, wherein the server confirms a tendency of odor diffusion using actual weather conditions and three-dimensional wind fields through an odor diffusion modeling program.

3. The system of claim 2, wherein the server analyzes the information on odor diffused in the air using a CALPUFF modeling technique showing a complex terrain and a change in wind field.

4. The system of claim 1, wherein the server classifies smell and odor and analyzes characteristics of each of smell and odor through a smell prediction program.

5. The system of claim 4, wherein the server classifies types and intensities of the odor information through a random forest model.

6. The system of claim 1, wherein the server analyzes the information on odor diffused in the air by granting different reliabilities to a first odor information obtained from the fixed odor measuring device, a second odor information obtained from the vehicular odor measuring device and a third odor information obtained from the drone.

7. The system of claim 6, wherein the server grants a highest reliability to the first odor information, a medium reliability to the second odor information and a lowest reliability to the third odor information.

8. The system of claim 6, wherein the server increases the reliability of the third odor information when an air current measured at a position of the drone is a descending air current, and decreases the reliability of the third odor information when an air current measured at a position of the drone is an ascending air current.
